(19) United States
(12) Patent Application Publication
(10) Pub. No.: US 2018/0133131 A1
(43) Pub. Date: May 17, 2018

(54) SKIN REJUVENATING COMPOSITIONS

(71) Applicant: ARIIX, Bountiful, UT (US)

(72) Inventors: Deanna Latson, Encinitas, CA (US); Tom Jackson, Murray, UT (US)

(21) Appl. No.: 15/812,310

(22) Filed: Nov. 14, 2017

Related U.S. Application Data
(60) Provisional application No. 62/421,542, filed on Nov. 14, 2016.

(51) Int. Cl. A61K 8/25; A61Q 19/08; A61K 8/64

(52) U.S. Cl. CPC A61K 8/25; A61K 2800/5922; A61K 8/64; A61Q 19/08

(57) ABSTRACT

A skin rejuvenating composition formulated to provide a first mode of action for providing relatively immediate skin tightening and wrinkle reducing effects upon application of the skin rejuvenating composition to a targeted skin region. The skin rejuvenating composition is also formulated to provide a second mode of action for providing cumulative and relatively more long-term wrinkle reduction effects, in addition to the more immediate effects. The dual modes of action work together to synergistically reduce the visibility of wrinkles to a greater degree than when either component is used in isolation.

### CROSS-REFERENCE TO RELATED APPLICATIONS

This application claims priority to and the benefit of U.S. Provisional Application Ser. No. 62/421,542, filed Nov. 14, 2016 and titled “SKIN REJUVENATING COMPOSITIONS,” the entirety of which is incorporated herein by this reference.

### BACKGROUND

Various skin rejuvenation techniques are used with the aim of rejuvenating a youthful appearance to the face or other dermal areas. On the face, localized facial concerns such as wrinkles, pigmentation defects, scars, and looseness are often the target of treatment. Some rejuvenation procedures involve the injection of material, such as Botox, dermal fillers, or collagen, into the targeted skin area. Other products are intended for topical application, and include those marketed to reduce or prevent signs of skin aging.

However, limitations to these compositions and methods exist. For example, injection of material into the dermal tissue can be painful. Otherwise suitable subjects are prone to forego such procedures due to an aversion to needles, anxiety about the associated pain, or the relatively high expense of the procedures. Products intended for topical application are less invasive. However, the actual anti-wrinkle results of such products are often less than desired by the user. Accordingly, there is a long felt and ongoing need for effective skin rejuvenating compositions.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

compositions which may be topically applied to dermal tissue in order to effectively provide one or more cosmetic benefits to the dermal tissue. At least some of the embodiments described herein function to reduce lines/wrinkles of dermal tissue to which the composition is applied. One or more of the embodiments described herein include a dual mode of action for providing the reduction in wrinkles, with a first mode of action providing relatively immediate skin tightening and wrinkle reducing results, and a second mode of action providing a more long-term wrinkle reduction effect upon continued application of the composition. The dual modes of action work together to synergistically reduce the visibility of wrinkles to a greater degree than when either component is used in isolation.

In some embodiments, a skin rejuvenating composition includes: (1) a skin tightening component formulated to provide a relatively immediate/instantaneous (e.g., within seconds or minutes) skin tightening effect upon application; (2) a skin restoring component formulated to provide cumulative long-term effects (e.g., apparent over weeks or months of continued use) in reducing wrinkle visibility upon application; and (3) a carrier.

In some embodiments, the skin tightening component includes silicates provided in the composition at a level of about 1% to about 12%, or at a level of about 2% to about 10%, or more preferably at about 4% to about 8%, or even more preferably at about 5.5% to about 6.75% by weight of the composition. Compositions utilizing such ranges were found to provide desired skin tightening effects without leaving an undesirable chalky or white residue at the application site. Further, inclusion of silicates within the foregoing ranges provided manufacturing and stability benefits by keeping the silicates within levels where they could be maintained in solution.

The combination of silicates may include sodium silicate and magnesium aluminum silicate. In some embodiments, the sodium silicate and magnesium aluminum silicate make up substantially all of the skin tightening component. In some embodiments, the ratio of magnesium aluminum silicate to sodium silicate in the skin tightening component is about 0.2 to about 2, or about 0.4 to about 1.5, or more preferably is about 0.5 to about 1.2, or even more preferably about 0.65 to about 1. For example, the skin tightening component may include sodium silicate at about 2% to about 5%, or more preferably at about 3% to about 3.75%, and may include magnesium aluminum silicate at about 1.5% to about 4.5%, or more preferably at about 2.5% to about 3%.

Certain embodiments are formulated to achieve a pH of about 9.5 to about 12.0, or more preferably about 10.4 to about 11.4. Values for the pH of the composition within the foregoing levels were found to maintain the silicates in solution and provide effective product stability.

In certain embodiments, the skin restoring component includes a suitable form of octapeptide-3, also known as SNAP-8. The octapeptide-3 may be provided as a peptide complex containing, from N terminus to C terminus, glutamic acid-glutamic acid-methionine-glutamine-arginine-arginine-aspartic acid, such as shown in Formula 1.

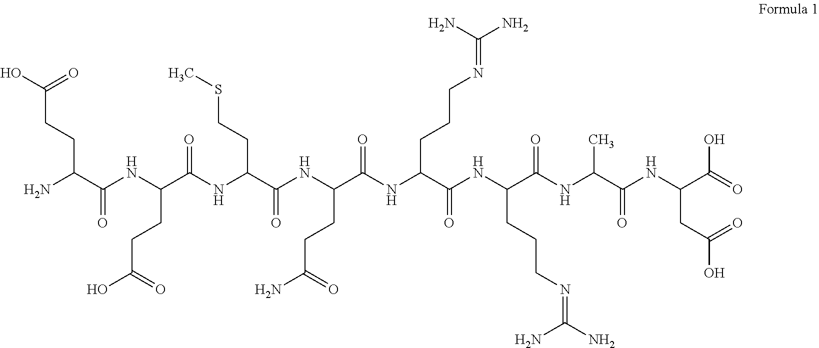 [SMILES: CSCCC(NC(=O)C(CCC(=O)O)NC(=O)C(N)CCC(=O)O)C(=O)NC(CCC(N)=O)C(=O)NC(CCCN=C(N)N)C(=O)NC(CCCN=C(N)N)C(=O)NC(C)C(=O)NC(CC(=O)O)C(=O)O]

In some embodiments, the octapeptide-3 of Formula 1 may include one or more modifications to promote or increase stability, shelf life, biological activity, cell permeability, resistance to enzymatic degradation, and/or other desired properties. Such modifications may include, for example, modifications to one or both termini to reduce the charge of the overall peptide, such as an acetylated N-terminus and/or an amidated C-terminus.

In some embodiments, the octapeptide-3 is provided at a concentration of about 0.001% to about 0.01%, or about 0.0015% to about 0.005%, or about 0.002% to about 0.0035%, or more preferably at about 0.0025% to about 0.003%. Inclusion of octapeptide-3 at levels within the foregoing ranges beneficially provided a cumulative effect in reducing wrinkle visibility over time.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### DETAILED DESCRIPTION

### Introduction

Certain embodiments described herein are directed to skin serum compositions which may be topically applied to dermal tissue in order to effectively provide one or more cosmetic benefits to the dermal tissue. At least some of the embodiments described herein function to reduce lines/wrinkles of dermal tissue to which the composition is applied. One or more of the embodiments described herein include a dual mode of action for providing the reduction in wrinkles, with a first mode of action providing relatively immediate skin tightening and wrinkle reducing results, and a second mode of action providing a more long-term wrinkle reduction effect upon continued application of the composition.

In some embodiments, a skin rejuvenating composition includes: (1) a skin tightening component formulated to provide a relatively immediate/instantaneous (e.g., within seconds or minutes) skin tightening effect upon application; (2) a skin restoring component formulated to provide cumulative long-term effects (e.g., apparent over weeks or months of continued use) in reducing wrinkle visibility upon application; and (3) a carrier.

Throughout the description, exemplary embodiments are described as “serums.” It will be understood, however, that alternative compositional forms may be formulated using similar components and principles described herein. For example, one of ordinary skill in the art will understand, in light of the disclosure presented herein, that at least some embodiments may be formulated as gels, creams, lotions, pastes, low viscosity washes, or other compositional forms suitable for topical administration to a targeted region of epidermal tissue. In addition, although most of the examples described herein in the context of application to the face, it will be understood that such compositions may be applied to other regions of skin where one or more of the effects of the compositions are desired.

Unless specifically described otherwise, concentrations and amounts of different components are given on a per weight basis relative to the total weight of the composition.

As used herein, the terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

### First Mode of Action

In presently preferred embodiments, the skin tightening component includes a combination of silicates formulated to provide the relatively immediate skin tightening effects of the composition. In some embodiments, the silicates are provided in the composition at a level of about 1% to about 12%, or at a level of about 2% to about 10%, or more preferably at about 4% to about 8%, or even more preferably at about 5.5% to about 6.75%.

When provided in amounts within the foregoing ranges, the silicates were found to provide effective skin tightening effects while minimizing or avoiding limitations often accompanying use of silicates. For example, as explained in more detail below, inclusion of silicates within the foregoing ranges provided manufacturing and stability benefits by keeping the silicates within levels where they could be maintained in solution. In contrast, inclusion of silicates at higher levels progressively increases the risk of precipitation of the silicates in solution, reducing the effectiveness and the shelf life of the product. On the other hand, inclusion of silicates at lower levels limits or removes the immediate skin tightening activity of the composition. Utilizing silicates in amounts within the foregoing ranges was thus found to optimize the balance between effective skin tightening effects and manufacturability/stability.

In addition, compositions utilizing such ranges were found to provide desired skin tightening effects without leaving a chalky or white residue at the application site. This type of residue can be highly undesirable. For instance, potential users who would otherwise benefit from use of a skin rejuvenating composition may forego its use because a highly visible residue is deemed not to be worth the benefits of the composition.

Any combination of cosmetically appropriate silicates may be utilized. In presently preferred embodiments, the combination of silicates includes sodium silicate and magnesium aluminum silicate. In some embodiments, the sodium silicate and magnesium aluminum silicate make up substantially all of the skin tightening component.

In some embodiments, the ratio of magnesium aluminum silicate to sodium silicate in the skin tightening component is about 0.2 to about 2, or about 0.4 to about 1.5, or more preferably is about 0.5 to about 1.2, or even more preferably about 0.65 to about 1. For example, the skin tightening component may include sodium silicate at about 2% to about 5%, or more preferably at about 3% to about 3.75%, and may include magnesium aluminum silicate at about 1.5% to about 4.5%, or more preferably at about 2.5% to about 3%. Inclusion of these particular types of silicates in these proportions was beneficially found to optimize the skin tightening effects of the composition while also limiting the potential manufacturing, stability, and residue-forming issues of the composition.

It was beneficially found that providing silicates in amounts and proportions within the foregoing ranges resulted in a final composition having effective skin tightening effects as well as providing a workable pH and viscosity. For example, a pH of the final composition of about 9.5 to about 12.0, or more preferably about 10.4 to about 11.4, was found to maintain the silicates in solution. In addition, providing silicates according to the foregoing ranges was found to provide an effective skin tightening component for providing relatively immediate skin tightening effects while avoiding the appearance of a white or chalky residue otherwise frequently resulting from application of silicate containing compositions.

### Second Mode of Action

In presently preferred embodiments, the skin restoring component includes a suitable form of octapeptide-3, also known as SNAP-8. Octapeptide-3 is a peptide complex including the amino acids alanine, arginine, aspartic acid, glutamic acid, glutamine, and methionine. In some embodiments, the octapeptide-3 is provided as a peptide complex containing, from N terminus to C terminus, glutamic acid-glutamic acid-methionine-glutamine-arginine-arginine-aspartic acid, such as shown in Formula 1.

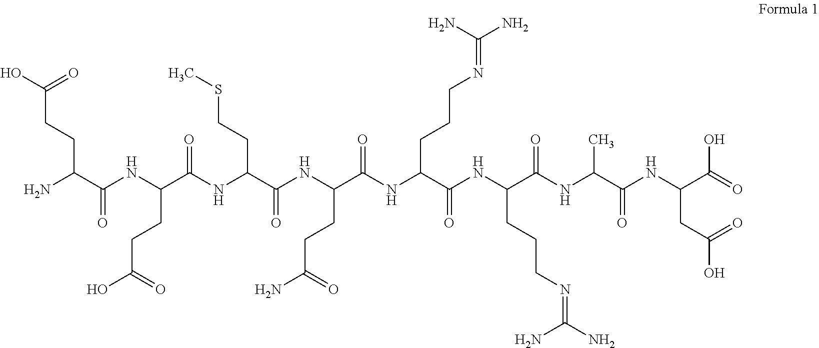 [SMILES: CSCCC(NC(=O)C(CCC(=O)O)NC(=O)C(N)CCC(=O)O)C(=O)NC(CCC(N)=O)C(=O)NC(CCCN=C(N)N)C(=O)NC(CCCN=C(N)N)C(=O)NC(C)C(=O)NC(CC(=O)O)C(=O)O]

In some embodiments, the octapeptide-3 of Formula 1 may include one or more modifications to promote or increase stability, shelf life, biological activity, cell permeability, resistance to enzymatic degradation, and/or other desired properties. Such modifications may include, for example, modifications to one or both termini to reduce the charge of the overall peptide, such as an acetylated N-terminus and/or an amidated C-terminus. Formula 2 shown below includes both an acetylated N-terminus and an amidated C-terminus.

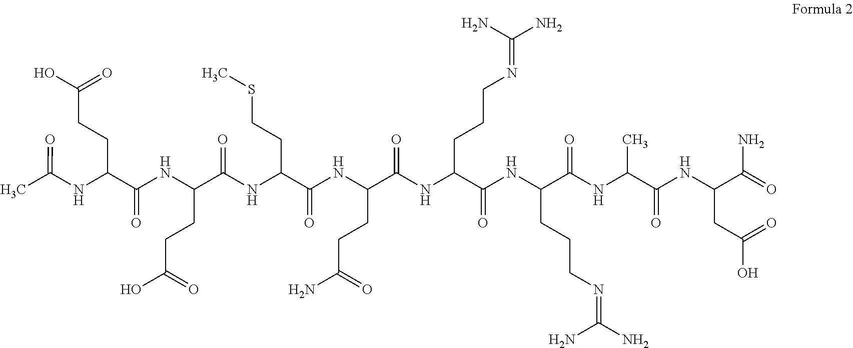 [SMILES: CSCCC(NC(=O)C(CCC(=O)O)NC(=O)C(CCC(=O)O)NC(C)=O)C(=O)NC(CCC(N)=O)C(=O)NC(CCCN=C(N)N)C(=O)NC(CCCN=C(N)N)C(=O)NC(C)C(=O)NC(CC(=O)O)C(N)=O]

The embodiment of Formula 2 is an octapeptide-3 in the form of acetyl octapeptide-3. The acetylated octapeptide-3 may be formed, for example, as a reaction product between acetic acid and octapeptide-3. Some embodiments may additionally or alternatively include a reaction product between octapeptide-3 and another suitable reactant (e.g., another suitable organic acid).

In some embodiments, the octapeptide-3 is provided at a concentration of about 0.001% to about 0.01%, or about 0.0015% to about 0.005%, or about 0.002% to about 0.0035%, or more preferably at about 0.0025% to about 0.003%. Inclusion of octapeptide-3 at levels within the foregoing ranges beneficially provided a cumulative effect in reducing wrinkle visibility over time.

Inclusion of octapeptide-3 at levels within the foregoing ranges was shown to provide a synergistic anti-wrinkle effect with the skin tightening component of the overall skin rejuvenating composition. Compositions described herein were therefore capable of providing effective anti-wrinkle effects in both near-term immediate effects and in long-term cumulative effects.

### Carrier Component

In some embodiments, the carrier component includes mostly water. For example, water may be included at about 60 to 90% of the total composition, or about 70 to 80% of the total composition. Water levels may be higher or lower than the foregoing ranges depending on particular application needs or desired compositional forms (e.g., creams, lotions, gels, etc.).

Carrier embodiments may also include, for example, one or more botanical blends, skin soothing agents, astringents, fragrance, coloring agents, alcohols, ketones, esters, plant oils, essential oils, mineral oils, triglycerides, ethers, organic solvents, glycols, glycerin, polyols, polyphenols, 1,3-propandiol, petroleum jelly, waxes, stearic acid, surfactants, or other desired ingredients suitable for cosmetic application to the skin.

For example, some embodiments may include one or more polyphenols (e.g., arjuna bark extract, grape seed extract, etc.) which are particularly advantageous in applications to dermal tissue. Additionally, or alternatively, the carrier may include one or more of triglycerides (e.g., grape seed oil, coconut oil, and the like), terpenes, other oils, amine compounds (such as mono-, di-, and tri-ethanol amine), and carbohydrates (such as sucrose, fructose, and higher polymers).

The carrier may additionally or alternatively be formulated as an emulsion, and may include liposomes, creams, and/or other like components. For example, the carrier may be formulated as a cream suitable for dermal application, such as a cream formed with a stearic acid base optionally containing oils such as coconut or olive oil, grape seed oil, vitamin E oil, and/or an emulsifying wax.

Gels known in the art may also be used as carriers, such as gels containing one or more of the foregoing liquid components together with known gelling agents.

The utilization of gels, creams, serums, and the like that are readily absorbed through the dermal layer further facilitates transport of the separate components of the skin rejuvenating composition into that dermal layer where the immediate and cumulative anti-wrinkle effects of the composition can be achieved.

### Methods of Administration

A method for reducing the appearance of wrinkles on a subject includes: (1) applying an effective amount of any of the skin rejuvenating compositions described herein to a targeted skin region of the subject; and (2) the skin rejuvenating composition reducing the appearance of wrinkles at the targeted skin region. The application is preferably performed repeatedly. For example, the composition may be applied daily (e.g., every morning or night) or twice daily (e.g., every morning and night), though other application schedules may also be utilized.

As explained above, the skin rejuvenating compositions provide effective reduction in wrinkle appearance in a relatively immediate fashion (e.g., under a minute, within about 5 minutes, within about 10 minutes, within about 20 minutes, within about an hour) while also providing a more long-term cumulative reduction in wrinkle appearance as the composition is continuously applied (e.g., for 5 days or more, 10 days or more, 2 weeks or more, 4 weeks or more, 6 weeks or more). In at least some implementations, the overall reduction in wrinkle appearance is enhanced by the synergistic relationship between the more immediate mode of action and the long-term, cumulative mode of action.

### EXAMPLES

Thirty-two subjects participated in a study testing a skin rejuvenation composition formulated according to the embodiments described herein. No adverse events or irritation was observed on any subject during the course of the study.

### Example 1

Crow's feet fine lines/wrinkles of the subjects were evaluated at different time points during continuous use of the composition. Table 1 illustrates the results of the testing as compared to an initial baseline measurement. Scores were provided by a trained technician during an evaluation of the face of each subject for crow's feet and fine lines/wrinkles.

TABLE 1
Technician Evaluation

| | | | Mean % | % of Subjects |
|---|---|---|---|---|
| | Mean Score ± | | Change from | with |
| | S.D. | p-value | Baseline | Improvement |
| Baseline | 5.8 ± 0.7 | — | — | — |
| Immediate | 5.3 ± 0.7 | <0.0001 | −7.9% | 47% |
| Week 4 | 4.7 ± 0.8 | <0.0001 | −17.9% | 84% |
| Week 6 | 4.3 ± 1.0 | <0.0001 | −26.8% | 97% |

The results show highly significant improvements, in both the degree of wrinkle reduction and in number of subjects showing improvements, when compared to baseline.

### Example 2

As in Example 1, crow's feet fine lines/wrinkles of the subjects were evaluated at different time points during continuous use of the composition. Table 2 illustrates the results of the testing as compared to an initial baseline measurement. In this Example, digital images were analyzed using image analysis software to provide the scores.

TABLE 2
Image Analysis

| | | | Mean % | % of Subjects |
|---|---|---|---|---|
| | Mean Score ± | | Change from | with |
| | S.D. | p-value | Baseline | Improvement |
| Baseline | 461.3 ± 71.7 | — | — | — |
| Immediate | 405.1 ± 33.6 | <0.0001 | −10.8% | 88% |
| Week 4 | 370.1 ± 37.3 | <0.0001 | −17.7% | 94% |
| Week 6 | 348.9 ± 28.5 | <0.0001 | −22.5% | 97% |

As in the technician evaluation of Example 1, the computer-automated results show highly significant improvements, in both the degree of wrinkle reduction and in number of subjects showing improvements, when compared to baseline.

What is claimed is:

1. A skin rejuvenating composition for reducing the appearance of wrinkles upon application to the skin of a subject, the composition comprising:
a skin tightening component formulated to provide relatively instantaneous skin tightening upon application;
a skin restoring component formulated to provide cumulative long-term effects in reducing wrinkle visibility; and
a carrier.

2. The composition of claim 1, wherein the skin tightening component includes silicates provided in the composition at a level of about 1% to about 12% by weight of the composition.

3. The composition of claim 2, wherein the skin tightening component includes sodium silicate and magnesium aluminum silicate.

4. The composition of claim 3, wherein the ratio of magnesium aluminum silicate to sodium silicate in the skin tightening component is about 0.2 to about 2.

5. The composition of claim 1, wherein the pH of the composition is about 9.5 to about 12.0.

6. The composition of claim 1, wherein the pH of the composition is about 10.4 to about 11.4.

7. The composition of claim 1, wherein the composition formulated so as to avoid the appearance of a white or chalky residue when applied to skin.

8. The composition of claim 1, wherein the skin restoring component includes octapeptide-3.

9. The composition of claim 8, wherein the octapeptide-3 is acetylated and/or amidated.

10. The composition of claim 8, wherein the octapeptide-3 is formulated according to Formula 1:

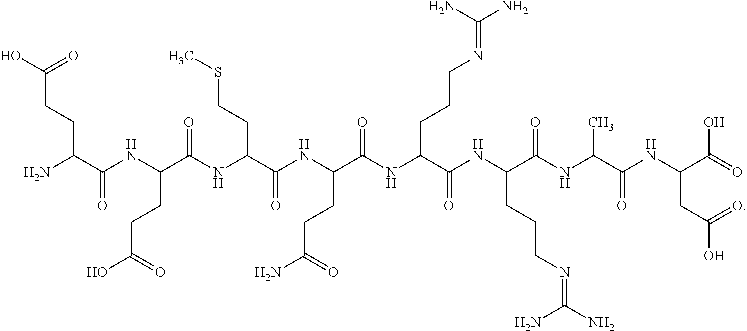 [SMILES: CSCCC(NC(=O)C(CCC(=O)O)NC(=O)C(N)CCC(=O)O)C(=O)NC(CCC(N)=O)C(=O)NC(CCCN=C(N)N)C(=O)NC(CCCN=C(N)N)C(=O)NC(C)C(=O)NC(CC(=O)O)C(=O)O]

11. The composition of claim 9, wherein the octapeptide-3 is formulated according to Formula 2:

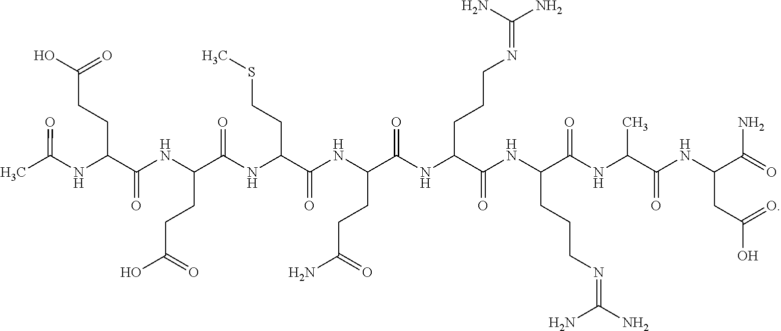 [SMILES: CSCCC(NC(=O)C(CCC(=O)O)NC(=O)C(CCC(=O)O)NC(C)=O)C(=O)NC(CCC(N)=O)C(=O)NC(CCCN=C(N)N)C(=O)NC(CCCN=C(N)N)C(=O)NC(C)C(=O)NC(CC(=O)O)C(N)=O]

12. The composition of claim 8, wherein the octapeptide-3 is provided at a concentration of about 0.001% to about 0.01% by weight of the composition.

13. The composition of claim 8, wherein the octapeptide-3 is provided at a concentration of about 0.002% to about 0.0035% by weight of the composition.

14. The composition of claim 1, wherein the carrier includes water at 60 to 90% by weight of the composition.

15. The composition of claim 1, wherein the composition is formulated as a serum.

16. A skin rejuvenating composition for reducing the appearance of wrinkles upon application to the skin of a subject, the composition comprising:
a skin tightening component formulated to provide relatively instantaneous skin tightening upon application;
wherein the skin tightening component includes silicates, the silicates including sodium silicate and magnesium aluminum silicate, provided in the composition at a level of about 1% to about 12% by weight of the composition to avoid the appearance of a chalky residue when applied to the skin;
a skin restoring component formulated to provide cumulative long-term effects in reducing wrinkle visibility;
wherein the skin restoring component includes octapeptide-3 at a concentration of about 0.002% to about 0.0035% by weight of the composition; and
a carrier,
wherein the composition has a basic pH.

17. The composition of claim 16, wherein the ratio of magnesium aluminum silicate to sodium silicate in the skin tightening component is about 0.2 to about 2.

18. A method for reducing the appearance of skin wrinkles on a subject, the method comprising:
applying an effective amount of a skin rejuvenating composition to a targeted skin region of the subject; the skin rejuvenating composition including—
a skin tightening component formulated to provide relatively instantaneous skin tightening upon application;
a skin restoring component formulated to provide cumulative long-term effects in reducing wrinkle visibility; and
a carrier; and
the skin rejuvenating composition reducing the appearance of wrinkles at the targeted skin region.

19. The method of claim 18, wherein the application is performed repeatedly at about once or twice a day for a time period of about 1 to 6 weeks.

20. The method of claim 18, wherein the skin tightening component includes silicates, the silicates including sodium silicate and magnesium aluminum silicate, provided in the composition at a level of about 1% to about 12% by weight of the composition to avoid the appearance of a chalky residue when applied to the skin, and wherein the skin restoring component includes octapeptide-3 at a concentration of about 0.002% to about 0.0035% by weight of the composition.
